(19) United States
(12) Patent Application Publication
(10) Pub. No.: US 2021/0393022 A1
(43) Pub. Date: Dec. 23, 2021

(54) CLOUD PLATFORM AND SYSTEM FOR COSMETICS PREPARATION

(71) Applicant: LONGTERM CONCEPT INTERNATIONAL PTE. LTD., Singapore (SG)

(72) Inventors: CHENG YU SHIH, Zhonghe Dist., New Taipei City (TW); YEE ANN HO, Singapore (SG)

(21) Appl. No.: 16/908,203

(22) Filed: Jun. 22, 2020

(51) Int. Cl. A45D 44/00; G06Q 30/06; G16H 10/20; G16H 50/20; G16H 50/50

(52) U.S. Cl. CPC A45D 44/005; G06Q 30/0621; G06Q 30/0641; G06Q 30/0631; G16H 50/20; G16H 50/50; G16H 10/20

(57) ABSTRACT

A cloud platform and system for cosmetics preparation, the cloud platform includes a database for storing at least one user's information, a storage for saving a number of predetermined formulas, and a user interface offering the user to choose one of the predetermined formulas and/or to create at least one of the personal formula and/or to create at least one of the personal formula in order to customize cosmetics living up to user's expectations within friendly accessibility and enjoyable experience.

### BACKGROUND OF THE INVENTION

### 1. Field of the Invention

This disclosure relates to a cloud platform and system for cosmetics preparation responsible for cosmetics formulation according to personal formula.

### 2. Description of the Related Art

As skin care has formed a habit in daily routine in order to keep a finest facial condition, the chase of cosmetics with proportional-right ingredients put the users in dilemma surrounded by a variety of selections. However, the cosmetics for sale filled in uniformed compositions can no longer keep up with the emerging demands in order to keeping the faces always in finest conditions as long as the users still put on the cosmetics with ingredients haven't been finely tuned to fit the skins susceptive to the slightly-changed on influence factors, such as the weather, temperament and environment . . . etc., per days even per hours.

Besides, the existing cosmetic preparation apparatuses are equipped with single-operational interface in need for users' acquaintance before producing cosmetics formulas suitable for personal use which is reckoned as boring and uninteresting because the of lack of users' interaction.

### SUMMARY OF THE INVENTION

In order to manage aforementioned defects, this disclosure provides a cloud platform and system for cosmetics preparation living up to individuals' requirement and preferences.

Based on abovementioned goal, this disclosure offers the cloud platform in connection to a terminal device and/or a cosmetic formulating apparatus. The cloud platform comprises a database for storing at least one user's information, a storage for saving a number of predetermined formulas, and a user interface for creating at least one user's information in connection to the storage for selecting at least one of the predetermined formulas and/or saving at least one personal formula back to the storage corresponding to at least one user's information.

Preferably, the user interface enables the user to make public of at least one of the personal formulas. In addition, the user's information includes the basic physiological information, environment information, lifestyle and activities, health status and mental condition.

Wherein, the cloud platform comprises a computing unit which prompts one of the predetermined formulas with time duration as recommendation after evaluation.

Wherein, the terminal device consists of an input unit and a display. The cosmetic formulating apparatus includes a modulating unit for receiving one of the predetermined formulas or at least one of the personal formulas before sending for cosmetic preparation by the modulating unit. The terminal device may be further installed with a validation unit to examine the authenticity of the cosmetic ingredients.

Still preferably, the cosmetic formulating apparatus has a plurality of cartridges of cosmetic ingredients and outputs the cosmetics formulated from predetermined formulas or personal formula.

This disclosure further offers the cosmetics preparation system includes foresaid cloud platform and the cosmetic formulating apparatus to perform the cosmetics preparation on receiving predetermined formulas or personal formula transmitted from the cloud platform.

The foresaid cosmetic formulating system may be accompanied with the terminal device in connection to the cloud platform for a handy activation of the cosmetics preparation.

By doing so, the users can obtain the predetermined formulas or create the personal formula to be shared with other users much increasing the accessibility and enjoyment with less fumble.

### BRIEF DESCRIPTION OF THE DRAWINGS

FIG. 1 illustrates a systematic diagram of cosmetics preparation system.

FIG. 2 illustrates another systematic diagram of cosmetics preparation system.

FIG. 3 illustrates a process diagram of cosmetics preparation system.

### DETAILED DESCRIPTION OF PREFERRED EMBODIMENTS

In order to articulate more clearly with the embodiments, structure and performance of this disclosure, the elaboration will be set forth along with the drawings.

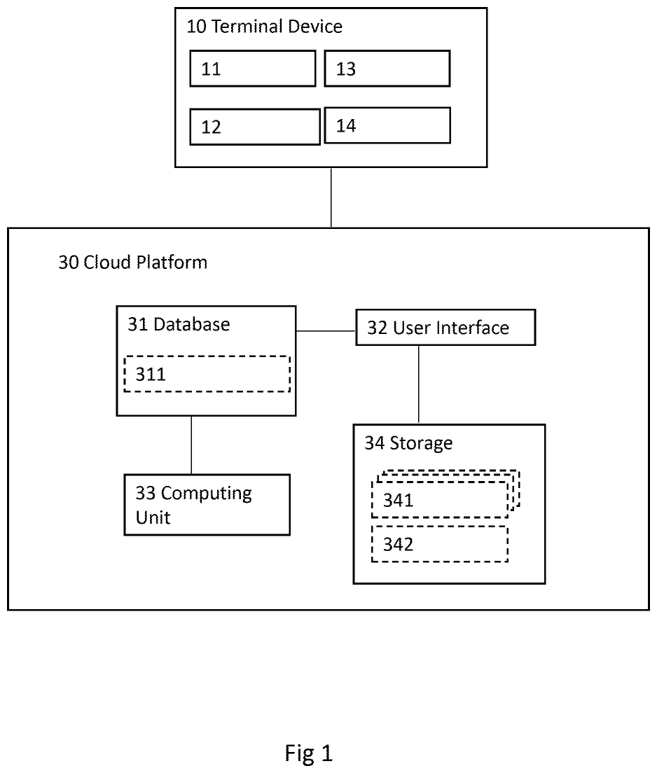

FIGS. 1, 2 and 3 illustrate an embodiment of a cosmetics preparation system providing the users with cosmetics of customized formulas including a cloud platform 30 in connection to a terminal device 10 and/or a cosmetic formulating apparatus 20, the cloud platform 30 including a database 31 storing at least one user's information 311, a storage 34 saving a number of predetermined formulas 341, and a user interface 32 linking to the storage 34 for retrieving one of the predetermined formulas 341 and/or creating at least one of the personal formulas 342 to be saved back to the storage 34 corresponding to at least one user's information 311. Besides, through setting on the user interface 32, the users can make public of at least one of the personal formulas 342 with the multiple users for formulation reference.

The cloud platform comprises a computing unit 33 which prompts one of the predetermined formulas 341 as recommendation with time duration after evaluating. Apart from offering an instant cosmetic formulating, the computing unit 33 also predicts recommended compositions for cosmetics preparation in advance of the users' travel or business trips much lessening the user's burden. The user's information 311 includes the basic physiological information, environment information, lifestyle and activities, health status and mental condition, wherein the physiological information includes either one of the following: the user's age, sex, skin type, height, weight or occupations. The environment information includes either one of the following: the temperature, moisture, present location and destination, UV index, rainfall, time of sunrise and sunset or air quality. Lifestyle and activities include either one of the following: users' diet preference and history, dietary program (meals and beverages), indoor and outdoor activities, working days and holidays or travel. Health status includes either one of the following: body mass index, menstrual cycle, skin condition or allergens. Mental condition includes either one of the following: users' sleep, Psychological test results, preferences or interests.

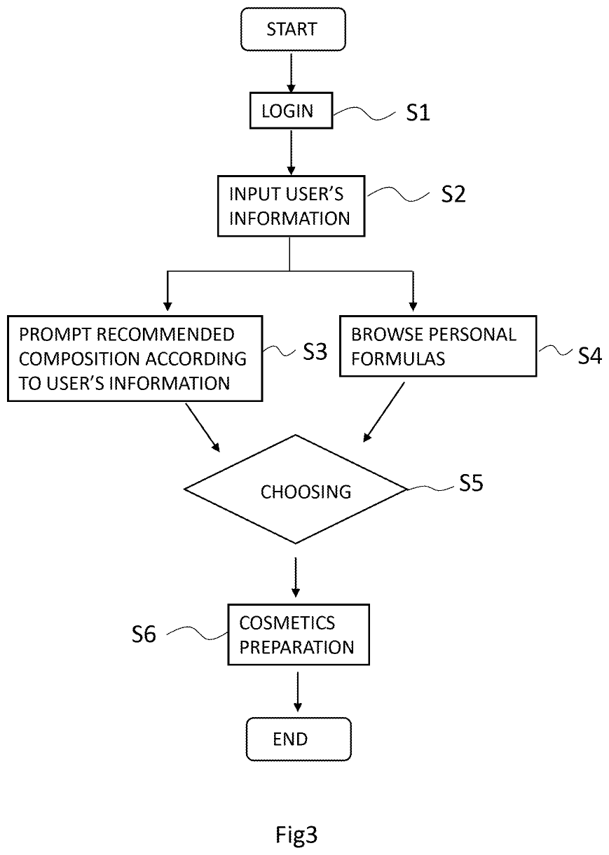

In this embodiment, the terminal device 10 links to the cloud platform 30 comprising an input unit 11, a display 12, a validation unit 14 and a modulating unit 13, shown as in FIG. 1 and FIG. 3. The users can log in the terminal unit 10 (step S1) and input the user's information 311 (step S2). The computing unit 33 evaluate the user's information and prompts one of the predetermined formulas 341 as recommendation (step S3), or user can browse the personal formulas (step S4). Then the modulating unit 13 performs the cosmetics preparation (step S6) from either one of the recommended compositions which has been calculated by the computing unit 33 according to user's information 311, or at least one of the personal formulas which has been made public by the users.

The terminal device 10 includes the validation unit 14 to verify the identification code ensuring the authenticity and the export quality of the cosmetic ingredients, and reminding the users of time duration to replace the cosmetics before the expiration date.

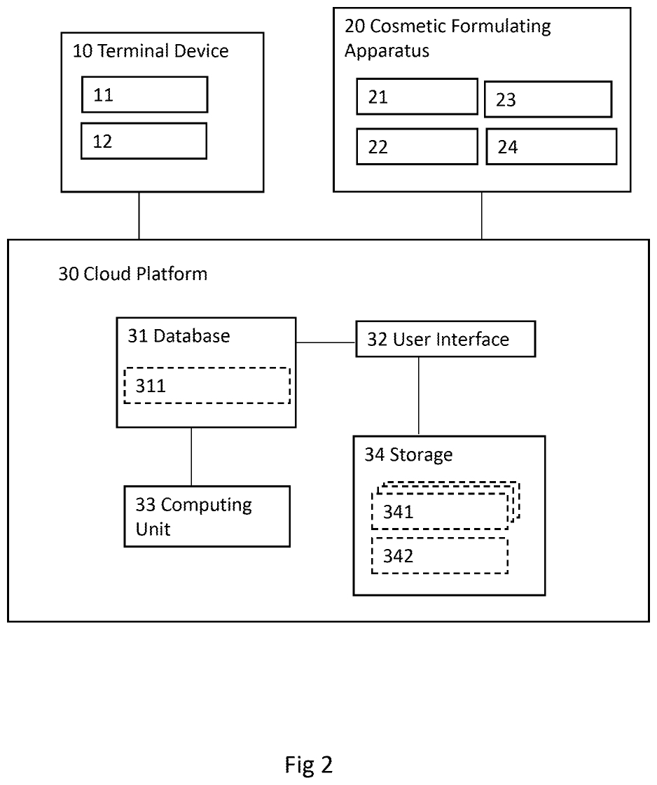

Furthermore, another embodiment of this disclosure demonstrates the cosmetics preparation system including the cloud platform 30 and the cosmetic formulating apparatus 20, shown as in FIG. 2. The cosmetic formulating apparatus 20 performs the cosmetics preparation on receiving either one of the predetermined formulas 341 or at least one of the personal formulas 342 transmitted from the cloud platform 30. Wherein, the system is equipped with the terminal device 10 in connection to the cloud platform 30 and/or the cosmetic formulating apparatus 20.

Wherein, the terminal device 10 includes an output unit 11 and the display 12. The cosmetic formulating apparatus 20 consists of the input unit 21, the display 22, the modulating unit 23 and the validation unit 24. The cosmetic formulating apparatus 20 performs cosmetics preparation on receiving one of the predetermined formulas 341 or at least one of the personal formulas 342 transmitted from the terminal device 10. Wherein, the terminal device 10 may be a mobile device.

Furthermore, through linking to the cloud platform 30, the users can process cosmetics preparation by activating the cosmetic formulating apparatus 20 installed in public places, such as hotels, bathhouses or the stations, at any time or at different locations so as to access the familiar cosmetic formulas without hesitation while away from home.

1. A cloud platform for cosmetics, links to at least one terminal device and/or at least one cosmetic formulating apparatus comprises:
a database for saving at least one user's information;
a storage for serving a number of predetermined formulas; and
a user interface for creating at least one user's information in connection to the storage, and for choosing one of the predetermined formulas and/or for creating at least one personal formula corresponding to at least one of the user's information to save back to the storage.

2. The cloud platform for cosmetics as defined in claim 1, wherein the user's information includes a user's basic physiological information, an environment information, a lifestyle and activities, a health status or a mental condition.

3. The cosmetic cloud platform for cosmetics as defined in claim 2, which comprises a computing unit providing one of the predetermined formulas as a recommended composition according to the user's information after evaluating.

4. The cloud platform for cosmetics as defined in claim 3, wherein the recommended composition have time duration.

5. The cloud platform for cosmetics as defined in claim 1, wherein the user interface serves the users to make public of the at least one personal formula with the multiple users.

6. The cloud platform for cosmetics as defined in claim 5, wherein the terminal device includes an input unit and a display, the cosmetic formulating apparatus including a modulating unit, the modulating unit performing cosmetics preparation on receiving one of the predetermined formulas or the at least one personal formula transmitted by the terminal device.

7. The cloud platform for cosmetics as defined in claim 6, wherein the cosmetic formulating apparatus further includes a validation unit.

8. The cloud platform for cosmetics as defined in claim 1, wherein the cosmetic formulating apparatus has a number of cartridges of cosmetic ingredients and outputs cosmetics according to the recommended composition or the personal formula.

9. The cosmetics preparation system includes the cloud platform for cosmetics as defined in claim 1, and at least one formulating apparatus performing cosmetics preparation on the receiving one of the predetermined composition or the at least one personal formula transmitted from the cloud platform.

10. The cosmetics preparation system as defined in claim 9, which is equipped with the terminal device in connection to the cloud platform and/or cosmetic formulating apparatus.
